(19) United States
(12) Patent Application Publication
(10) Pub. No.: US 2008/0086098 A1
(43) Pub. Date: Apr. 10, 2008

(54) Cannula for dispensing fluid products for vaginal and anal applications

(76) Inventor: Antonio Fontana, (US)

Correspondence Address: BROWDY AND NEIMARK, P.L.L.C.;624 NINTH STREET, NW, SUITE 300, WASHINGTON, DC, 20001-5303, US

(21) Appl. No.: 11/578,143

(22) PCT Filed: Apr. 6, 2005
(86) PCT No.: PCT/EP05/51532

§ 371 Date: Aug. 6, 2007

(30) Foreign Application Priority Data
Apr. 8, 2004 (IT) MO2004A000073

(51) Int. Cl. A61M 31/00

(52) U.S. Cl. 604/275; 604/279

(57) ABSTRACT

A cannula (1) for dispensing fluid products for vaginal and anal applications comprising: an inner tubular body (2) with a handgrip (4); at least one straight longitudinal slot (5), which is formed on the lateral surface of the inner tubular body; an outer tubular body (6), which is open at its two opposite ends (6a, 6b) and is fitted substantially coaxially over the inner tubular body and is associated therewith with a rotary coupling; a helical guide (7), which is formed on the inner lateral surface of the outer tubular body; a plunger (8), with at least one pin (9), which is inserted so that it can slide in the straight longitudinal slot and has an end (9a) that extends beyond the straight longitudinal slot and mates with the helical guide; a covering jacket (10), fitted detachably on the outer tubular body; and tamper-resistant means (12) for sealing the covering jacket to the handgrip, the tamper-resistant sealing means comprising a plurality of bridges (20).

### TECHNICAL FIELD

The present invention relates to a cannula for dispensing fluid products for vaginal and anal applications, particularly for applying ointments, creams, medicinal pastes or the like.

### BACKGROUND ART

Cannulas for dispensing medicinal fluid products are known which are used in particular for vaginal and anal applications and generally are sold in packages together with tubes or bottles that contain one of said products.

Known cannulas are constituted by a cylinder that is suitable to contain the product and inside which a plunger, rigidly coupled to the end of a pusher rod, can slide.

The cylinder has an open end, which can be coupled to the dispensing outlet of the tube, in order to introduce in said cylinder the amount of product to be applied, and through which the introduced product is dispensed.

The opposite end of the cylinder is closed by a bottom provided with a hole, in which the pusher rod is inserted so that it can slide and which acts as an element for stopping the plunger in order to prevent its extraction.

The product introduced in the cannula is dispensed by acting on the pusher rod in the direction of the sliding of the plunger toward the open end of the cylinder.

Cannulas are also known which are constituted by a cylinder, in which the opposite ends are open and inside which a plunger is inserted so that it can slide, and by a pusher rod, which is separate from the plunger and can be coupled detachably thereto.

Abutment undercuts or shoulders are formed at the opposite ends of the cylinder and are suitable to stop the sliding of the plunger, preventing it from disengaging under the action of the pusher rod.

These cannulas can be sold in packages that contain a single pusher rod, a plurality of empty single-use cylinders to be used for the various applications, and one or more tubes of product.

In this case, one of the two ends of the cylinders can be coupled to the dispensing outlet of the tube to introduce the product in said cylinders, while the opposite end acts as a passage for the pusher rod.

As an alternative, these cannulas can be sold in packages that contain a single pusher rod and a plurality of cylinders that are already filled with the product to be applied and are provided, at their two opposite ends, with closure plugs, which are removed at the time of use.

These known cannulas, however, are not free from drawbacks, including the fact that they have significant dimensions that make them difficult and awkward to handle for users.

Another drawback of known cannulas, particularly those constituted by a rod that is detachably associable with the plunger, is that they are not ready for immediate use by users, as they require additional assembly operations.

Another drawback of known cannulas is that the sliding of the plunger under the thrust applied to the rod often causes uncontrolled dispensing of amounts of product that are unwanted, since they are excessive or insufficient for the actual requirements of use.

### DISCLOSURE OF THE INVENTION

The aim of the present invention is to eliminate the drawbacks noted above of known cannulas, by providing a cannula for dispensing fluid products for vaginal and anal applications that is compact, simple and straightforward to use for users, and allows controlled and gradual dispensing of the product.

Within this aim, an object of the present invention is to provide a structure that is simple, relatively easy to provide in practice, safe in use, effective in operation, and has a relatively low cost.

This aim and this and other objects that will become better apparent hereinafter are achieved by the present cannula for dispensing fluid products for vaginal and anal applications, comprising: an inner tubular body for containing a fluid product, which is provided, at a first end, with an outlet for dispensing said product and, at a second end that lies opposite the first end, with a handgrip; at least one straight longitudinal slot, which is formed on the lateral surface of said inner tubular body; an outer tubular body, which is open at its two opposite ends and is fitted substantially coaxially over said inner tubular body and is associated therewith with a rotary coupling; a helical guide, which is formed on the inner lateral surface of said outer tubular body; a plunger, which is accommodated in said inner tubular body, with which it is associated so that it can slide axially, and is provided with at least one pin, which is inserted so that it can slide in said straight longitudinal slot and has an end that extends beyond said straight longitudinal slot and mates with said helical guide, the relative rotation of said inner tubular body and of said outer tubular body being suitable to move said plunger so that it slides along said inner tubular body to expel said product from said dispensing outlet; a covering jacket, which is provided with an open end and a closed end and is fitted detachably on said outer tubular body; and tamper-resistant means for sealing said covering jacket to said handgrip, characterized in that said tamper-resistant sealing means comprise a plurality of bridges, which have an end that is associated with said covering jacket and an opposite end that is associated with a band that is rigidly coupled to said handgrip, breakable regions being formed proximate to at least one of the opposite ends of said bridges.

### BRIEF DESCRIPTION OF THE DRAWINGS

Further characteristics and advantages of the present invention will become better apparent from the following detailed description of a preferred but not exclusive embodiment of a cannula for dispensing fluid products for vaginal and anal applications, illustrated by way of non-limiting example in the accompanying drawings, wherein:

FIG. 1 is a front view of the cannula according to the invention in the closed configuration for packaging;

FIG. 2 is a front view of the cannula according to the invention in the open configuration for use;

FIG. 3 is a schematic sectional view of the cannula according to the invention in the closed configuration for packaging;

FIG. 4 is an enlarged-scale view of a detail of FIG. 3;

FIG. 5 is a schematic view of the inner tubular body of the cannula according to the invention;

FIG. 6 is a partially sectional schematic view of the outer tubular body of the cannula according to the invention;

FIG. 7 is a schematic view of the covering jacket of the cannula according to the invention;

FIG. 8 is a partially sectional schematic view of a detail of the handgrip of the cannula according to the invention;

FIG. 9 is a schematic sectional view of the plunger of the cannula according to the invention;

FIG. 10 is a front view of the plunger of the cannula according to the invention.

### WAYS OF CARRYING OUT THE INVENTION

With reference to the figures, the reference numeral 1 generally designates a cannula for dispensing fluid products P for vaginal and anal applications, particularly for the application of ointments, creams, medicinal pastes or the like.

The cannula 1 comprises an inner tubular body 2 for containing the product P, which has, at a first end 2a, an outlet 3 for dispensing the product P and, at a second end 2b arranged opposite the first end, a handgrip 4; at least one and preferably two diametrically opposite longitudinal straight slots 5 are provided on the lateral surface of the inner tubular body 2.

An outer tubular body 6 is fitted substantially coaxially over the inner tubular body 2, is open at the two opposite ends 6a and 6b, and is associated with the inner tubular body 2 with a rotary coupling; a groove-shaped helical guide 7 runs between the two opposite ends 6a and 6b and is provided on the inner lateral surface of the outer tubular body 6.

A plunger 8 is accommodated within the inner tubular body 2, with which it is associated so that it can slide axially; the plunger 8 is provided with at least one and preferably two mutually diametrically opposite pins 9, which are inserted so that they can slide in a respective slot 5 and have a respective end 9a that protrudes beyond the slots 5 in order to mate slidingly within the helical guide 7.

The cannula 1 further comprises a covering jacket 10 of the removable type, which is fitted over the outer tubular body 6; the jacket 10 is of the tubular type, with an open end 10a, for inserting and extracting the outer tubular body 6 in and from it, and an opposite end 10b, which is closed and at which there is a sealing portion 11 having an inside diameter that is substantially smaller than the outside diameter of the end 6a of the outer tubular body 6.

When the cannula 1 is in the closed packaging configuration, the sealing portion 11 adheres to the end 6a, applying thereto a slight pressure, which is suitable to avoid seepages of product P between the jacket 10 and the outer tubular body 6.

The handgrip 4 and the jacket 10 are anchored temporarily to each other by tamper-resistant sealing means 12.

The handgrip 4 comprises a tang 13, which is formed at the second end 2b of the inner tubular body 2 and protrudes beyond the outer tubular body 6, and a cap 14, which is fitted rigidly over the tang 13.

Between the tang 13 and the cap 14 there are means for locking relative rotation and means for locking the relative axial sliding of one with respect to the other.

The means for locking the relative rotation between the tang 13 and the cap 14 can be of the type of a side-fit coupling or the like, and can be constituted for example by a plurality of knurled portions 15, which lie longitudinally on at least one portion of the inner lateral surface of the cap 14 and engage corresponding knurled portions 16 formed on a respective portion of the outer lateral surface of the tang 13; advantageously, the portions are two and are mutually diametrically opposite.

The means for locking the relative axial sliding between the tang 13 and the cap 14 can be of the interlocking type and be constituted for example by a groove 17, which is formed on the outer lateral surface of the tang 13 and mates by interlocking with a corresponding ring 18 formed on the inner lateral surface of the cap 14.

An abutment 19 for the tang 13 is also formed inside the cap 14.

The cap 14 is thus rigidly coupled to the tang 13 and therefore to the inner tubular body 2.

The tamper-resistant sealing means 12 comprise a plurality of bridges 20, which are distributed, so as to be alternated with spacers 21, along the perimetric rim of the open end 10a of the jacket 10 and have an end that is rigidly coupled to said rim and an opposite end that is coupled temporarily, at respective tearable/breakable regions 22, to a band 23 that is suitable to be rigidly coupled to the handgrip 4.

The band 23 comprises a fixing flap 23a, which is rigidly coupled to the perimetric rim of the grip 4, for example by interlocking in a seat 24 formed along the perimetric rim of the cap 14, and an indicator flap 23b, which protrudes from the handgrip 4 (seat 24) to indicate that the cannula 1 has been opened.

As an alternative, the fixing flap 23a could be rigidly coupled to the handgrip 4 with systems other than interlocking, for example by adhesive bonding, welding, ultrasound welding, or others.

The step for assembling the jacket 10 on the outer tubular body 6 occurs by pushing; in particular, the band 23, coupled to the bridges 20, is pushed in the seat 24; the spacers 21 act, during this step, as buffers, reducing the flexing of the bridges 20 and therefore the risk of their breakage.

The cap 14 is extended by a tubular sealing tab 25, which covers a preset extent inside the jacket 10 and is suitable to prevent air, dust or other external agents from entering the jacket 10 through the gaps 26 left free between the bridges 20 and the spacers 21.

Shoulders 27, respectively for the abutment of the opposite ends 6a and 6b of the outer tubular body 6, are formed at the first end 2a and at the second end 2b of the inner tubular body 2.

The stroke of the plunger 8 is limited by retention grooves 28 for the pins 9, which are formed at at least one of the opposite ends of the slots 5 and substantially transversely thereto.

Conveniently, on the outer lateral surface of the jacket 10 and of the outer tubular body 6 there are grip recesses 29 of the corrugated or knurled type, which are suitable to ensure the manual grip of a user, preventing the user's fingers from slipping during the maneuvers for using the cannula 1 and in particular the relative rotation between the handgrip 4 and the jacket 10 to open the cannula 1 and between the handgrip 4 (rigidly coupled to the inner tubular body 2) and the outer tubular body 6, for dispensing the product P.

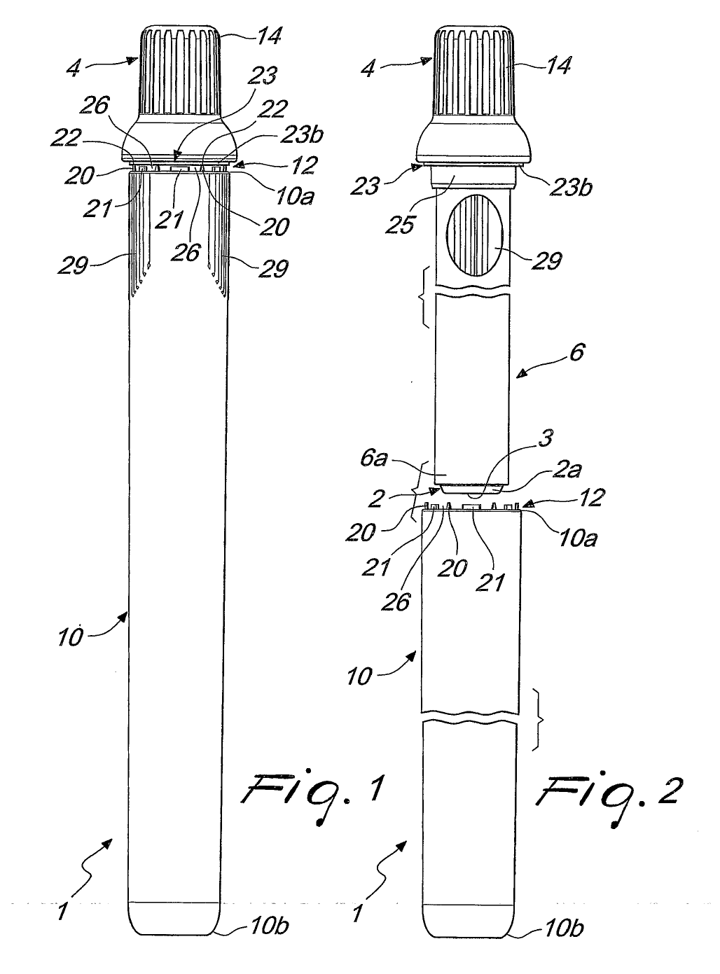

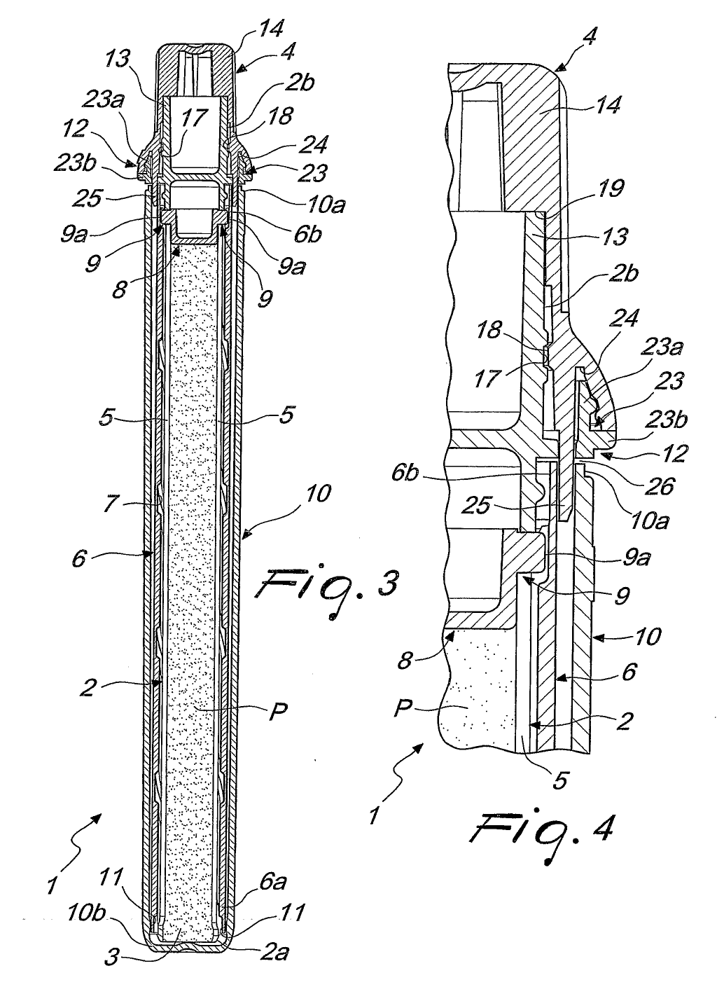

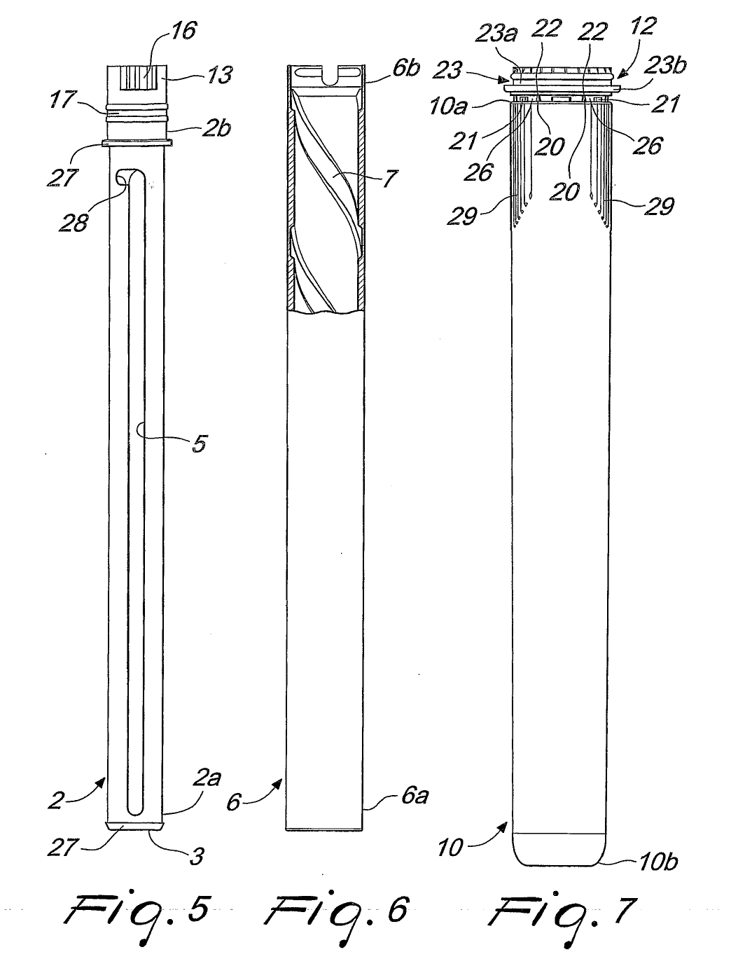

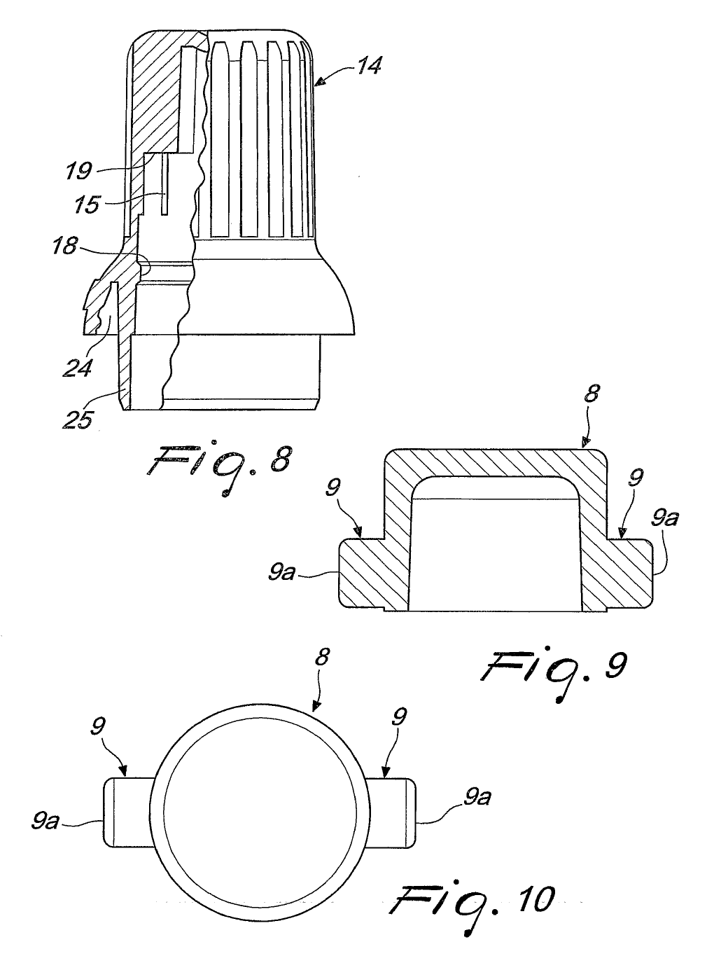

The cannula 1 is sold in the closed packaging configuration (FIGS. 1 and 3), filled with the product P; in this configuration, the jacket 10 is fitted over the outer tubular body 6 so as to close the dispensing outlet 3 and is anchored temporarily, by way of the tamper-resistant sealing means 12, to the handgrip 4, while the plunger 8 is arranged at the beginning of its stroke proximate to the tang 13.

To open the cannula 1, it is necessary to rotate the jacket 10 with respect to the handgrip 4, thus breaking the tearable/breakable regions 22 that temporarily couple the bridges 20 to the band 23, which is rigidly coupled to the handgrip 4; the jacket 10 is thus disengaged from the handle 4, the bridges 20 remain rigidly coupled to the jacket 10, and the strap 23 remains coupled to the cap 14 with the indicator flap 23b that protrudes therefrom to indicate that opening has occurred.

By extracting the jacket 10, the dispensing outlet 3 is connected to the outside and the cannula 1 is in the open configuration for use, ready for the application of the product P (FIG. 2).

For application, it is sufficient to act on the handgrip 4, keeping motionless the outer tubular body 6, in order to turn the inner tubular body 2 with respect to the outer tubular body 6; as a consequence of said relative rotation, the resulting force that acts on the pins 9, locked so as to slide along the slots 5 and along the helical guide 7, moves the plunger 8 so that it slides axially along the inner tubular body 2 in order to expel the product P from the dispensing outlet 3.

In practice it has been found that the described invention achieves the proposed aim and object.

The cannula according to the invention in fact is compact, particularly with respect to known cannulas provided with a pusher rod, making it easier to handle.

Moreover, the cannula according to the invention is simple and straightforward to use and, by way of the pressure applied to the plunger following the relative rotation imparted by the user to the inner tubular body with respect to the outer tubular body, allows better control and graduality of the dispensing of the product packaged therein.

The invention thus conceived is susceptible of numerous modifications and variations, all of which are within the scope of the appended claims.

All the details may further be replaced with other technically equivalent ones.

In practice, the materials used, as well as the shapes and the dimensions, may be any according to requirements without thereby abandoning the scope of the protection of the appended claims.

The disclosures in Italian Patent Application No. MO2004A000073 from which this application claims priority are incorporated herein by reference.

1. A cannula for dispensing fluid proucts for vaginal and anal applications, comprising: an inner tubular body for containing a fluid product, which is provided, at a first end, with an outlet for dispensing said product and, at a second end that lies opposite the first end, with a handgrip; at least one straight longitudinal slot, which is formed on the lateral surface of said inner tubular body; an outer tubular body, which is open at its two opposite ends and is fitted substantially coaxially over said inner tubular body and is associated therewith a rotary coupling; a helical guide, which is formed on the inner lateral surface of said outer tubular body; a plunger, which is accommodated in said inner tubular body, with which it is associated so that it can slide axially, and is provided with at least one pin, which is inserted so that it can slide in said straight longitudinal slot and has an end that extends beyond said straight longitudinal slot and mates with said helical guide, the relative rotation of said inner tubular body and of said outer tubular body being suitable to move said plunger so that it slides along said inner tubular body to expel said product from said dispensing outlet; a covering jacket, which is provided with an open end and a closed end and is fitted detachably on said outer tubular body; and tamper-resistant means for sealing said covering jacket to said handgrip, characterized in that said tamper-resistant sealing means comprise a plurality of bridges, which have an end that is associated with said covering jacket and an opposite end that is associated with a band that is rigidly coupled to said handgrip, breakable regions being formed proximate to at least one of the opposite ends of said bridges.

2. The cannula according to claim 1, characterized in that said bridges are distributed along the perimetric rim of said open end of the covering jacket.

3. The cannulas according to claim 1, characterized in that said breakable regions are formed proximate to the end of said bridges that is associated with said band.

4. The cannula according to claim 1, characterized in that said tamper-resistant sealing means comprise a plurality of spacers, which are distributed along said perimetric rim of the open end of the covering jacket and are alternated with said bridges.

5. The cannula according to claim 1, characterized in that said handgrip comprises a tang, which is formed at said second end of the inner tubular body and extends beyond said outer tubular body, and a cap, which is fitted rigidly over said tang, between said tang and said cap there being means for locking the relative rotation and means for locking the relative sliding of one with respect to the other.

6. The cannula according to claim 1, characterized in that said means for locking the relative rotation of said tang and said cap are of the type of a side-fit coupling or the like.

7. The cannula according to claim 1, characterized in that said means for locking the relative rotation of said tang and said cap comprise a plurality of knurled portions, which run longitudinally along at least one portion of the inner lateral surface of said cap and mesh with corresponding knurled portions formed on a respective portion of the outer lateral surface of said tang.

8. The cannula according to claim 1, characterized in that said means for locking the relative sliding of said tang and said cap are of the interlocking type.

9. The cannula according to claim 1, characterized in that said means for locking the relative sliding of said tang and said cap comprise at least one ring, which protrudes from said inner lateral surface of said cap and mates with an interlocking coupling with a corresponding annular groove formed on the outer lateral surface of said tang.

10. The cannula according to claim 1, characterized in that said band comprises a fixing flap, which is rigidly coupled to the perimetric rim of said cap, and an indicator flap, which protrudes from said cap.

11. The cannula according to claim 1, characterized in that a sealing portion is formed proximate to said closed end of said covering jacket and has an inside diameter that is substantially smaller than the outside diameter of said tubular body.

12. The cannula according to claim 1, characterized in that said cap is extended by a tubular sealing tab, which lies for a preset extent within said covering jacket and is suitable to prevent the penetration therein of external agents through the free gaps between said bridges and/or said spacers.

13. The cannula according to claim 1, characterized in that said first and second ends of the inner tubular body comprise abutment shoulders respectively for the opposite ends of said outer tubular body.

14. The cannula according to claim 1, characterized in that said straight longitudinal slots are two and are diametrically mutually opposite, said piston being provided with respective two of said pins, which are mutually diametrically opposite.

15. The cannula according to claim 1, characterized in that it comprises grooves for stopping the stroke of said plunger, which are formed at at least one of the opposite ends of said straight longitudinal slots in a direction that is substantially transverse thereto.

16. The cannula according to claim 1, characterized in that said helical guide is formed by a groove that lies between the opposite ends of said outer tubular body.

17. The cannula according to claim 1, characterized in that it comprises grip impressions, of the type with a corrugated or knurled surface or the like, which are formed on the outer lateral surface of said covering jacket and/or of said outer tubular body.

18. The cannulas according to claim 2, characterized in that said breakable regions are formed proximate to the end of said bridges that is associated with said band.

19. The cannula according to claim 18, characterized in that said tamper-resistant sealing means comprise a plurality of spacers, which are distributed along said perimetric rim of the open end of the covering jacket and are alternated with said bridges.

20. The cannula according to claim 19, characterized in that said handgrip comprises a tang, which is formed at said second end of the inner tubular body and extends beyond said outer tubular body, and a cap, which is fitted rigidly over said tang, between said tang and said cap there being means for locking the relative rotation and means for locking the relative sliding of one with respect to the other.
